(19) United States
(12) Patent Application Publication
(10) Pub. No.: US 2007/0270487 A1
(43) Pub. Date: Nov. 22, 2007

(54) Novel process and intermediates 085

(76) Inventors: Martin Hedberg, Sodertalje (SE); Marita Hogberg, Huddinge (SE); Weimin Tong, Huddinge (SE)

Correspondence Address: WHITE & CASE LLP;PATENT DEPARTMENT, 1155 AVENUE OF THE AMERICAS, NEW YORK, NY, 10036, US

(21) Appl. No.: 11/749,856

(22) Filed: May 17, 2007

Related U.S. Application Data
(60) Provisional application No. 60/801,555, filed on May 18, 2006.
(60) Provisional application No. 60/908,253, filed on Mar. 27, 2007.

(51) Int. Cl. A61K 31/165; A61K 31/325; A61K 31/137

(52) U.S. Cl. 514/485; 558/406; 514/617; 514/649; 560/29; 564/185; 564/348

(57) ABSTRACT

The present invention relates to a novel process suitable for large-scale production of phenyl propane derivatives as well as to novel intermediates in the process.

This application claims the priority benefit of U.S. Provisional Application No. 60/908,253, filed Mar. 27, 2007 and U.S. Provisional Application No. 60/801,555, filed May 18, 2006.

### FIELD OF THE INVENTION

The present invention relates to a novel process as well as to novel intermediates in the process.

### BACKGROUND

In Urbanski, T.; Lange, J. Rocznicki Chemii 1959, 33, 197, Makosza, M. Rocznicki Chemii 1969, 43, 333, Mndzboyan, Sh. L. et al. Zhurnal Organicheskoi Khimii 1982, 18, 1885, in EP 0428434 and in Mann, A. et al. J. Med. Chem. 1991, 34, 1307 the synthesis of racemic esters of 3-cyano-3-arylpropionic acids are described.

Miyake, A. et al. Takeda Kenkyushoho 1982, 41, 1, SU 363698, Jullian, V. et al. Synthesis 1997, 1091, WO 2000/002859, EP 0428434, EP 0559538 and WO 94/10146 disclose the synthesis of both racemic and optically pure 4-amino-3-aryl-butan-1-ols.

In U.S. Pat. No. 5,583,134, EP 0559538, EP 0474561 and in Karla R. et al. J. Med. Chem. 1999, 42, 2053 the syntheses of both racemic and optically pure (4-hydroxy-2-phenyl-butyl)-carbamic acid esters are described.

In Parker, J. S. et al. Org. Proc. Dev. 2003, 7, 67, WO 2004/110344, WO 2000/002859, Kubota, H. et al. Chem. Pharm. Bull. 1998, 46, 242, Jullian, V. et al. Synthesis 1997, 1091, WO 94/10146, and EP 0474561 describe the synthesis of both racemic and optically pure 4-methylamino-3-aryl-butan-1-ols.

In WO 2001/077069, WO 97/30989, Ohnmacht, C. J. et al. Bioorg. Med. Chem. 2004, 12, 2653, U.S. Pat. No. 5,236,921 and in WO 2002/026724 the syntheses of both racemic and optically pure N-(4-hydroxy-2-phenyl-butyl)-amides are described.

Makosza, M. and Marcinowicz, A. Synthesis 2001, 1311 and EP 0612716 describe methods for the preparation of racemic as well as enantiomerically pure 3-cyano-3-aryl-propionic acids. EP 0612716 also describes the synthesis of optically pure 4-amino-3-(3,4-dichlorophenyl)-butan-1-ol.

WO 00/02859 discloses the compounds 4-amino-3-(3,4-difluorophenyl)-1-butanol, 3-(3,4-difluorophenyl)-4-(ethoxycarbonylamino)-1-butanol and 3-(3,4-difluorophenyl)-N-methyl-4-amino-1-butanol.

Kubota H., Kafefuda A., Nagaoka H., Yamamoto O., Ikeda K., Takeuchi M., Shibanuma T., Isomura Y., Chemical & Pharmaceutical Bulletin 1998, 46 (2), 242-254 discloses the compounds 3-(4-fluorophenyl)-4-(methylamino)butan-1-ol, 3-(4-bromophenyl)-4-(methylamino)butan-1-ol and 4-(methylamino)-3-(4-methylphenyl)butan-1-ol.

The problem underlying the present invention was to find a novel process suitable for large-scale production of phenyl propane derivatives of formula (I) below.

### OUTLINE OF THE INVENTION

The present invention relates to a novel process for the manufacture of substituted phenyls of formula (I):

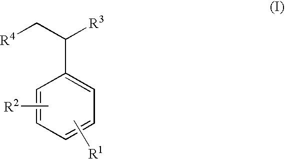

wherein

R¹ is selected from fluoro, bromo, iodo, C₁-C₁₀ alkyl, phenyl, C₃-C₆ cycloalkyl, trifluoromethyl, difluoromethyl and fluoromethyl;

R² is selected from hydrogen, fluoro, bromo, iodo, C₁-C₁o alkyl, trifluoromethyl, difluoromethyl and fluoromethyl;

R³ is CH₂NR₅R₆;

R⁴ is CH₂OH;

R⁵ and R⁶ are independently selected from hydrogen, methyl, COR⁸ and COOR⁹;

R ⁸ is

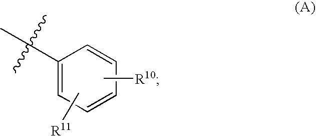

R⁹ is selected from C₁-C₄ alkyl;

R¹⁰ is selected from fluoro, chloro, bromo, iodo, C₁-C₁₀ alkyl, phenyl, C₃-C₆ cycloalkyl, trifluoromethyl, difluoromethyl and fluoromethyl;

R¹¹ is selected from fluoro, chloro, bromo, iodo, C₁-C₁₀ alkyl, phenyl and C₃-C₆ cycloalkyl.

with the proviso that R⁵ and R⁶ are not the same unless both R⁵ and R⁶ are hydrogen; and

with the further proviso that if one of R⁵ or R⁶ is COR⁸ then the other is methyl; and

with the further proviso that if one R⁵ or R⁶ is COOR⁹, then the other is hydrogen;

comprising the following steps:

Step 1:

(i) A compound of formula (II),

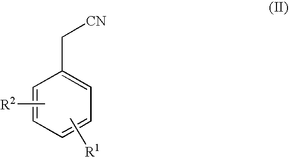

is reacted with a haloacetic acid or a salt or an ester thereof in a solvent in the presence of a base, whereby a compound of formula (III) is obtained

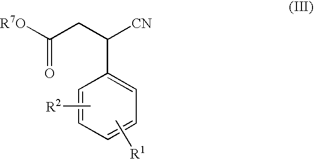

wherein

R⁷ is selected from tert-butyl, iso-butyl, iso-propyl and iso-amyl;

R¹ and R² are as defined for the compound of formula (I) above;

(ii) the compound of formula (III) is purified.

Step 2

(i) The compound of formula (III) obtained from Step (I) is reacted with a reducing agent in a solvent, whereby a compound of formula (IV) is obtained

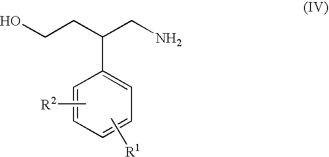

wherein R¹ and R² are as defined for the compound of formula (I) above;

(ii) the compound of formula (IV) is optionally purified.

Step 3

(i) A racemic compound of formula (IV) obtained in Step 2 is resolved into the corresponding (R)- and (S)-enantiomers, R-(IV) and S-(IV),

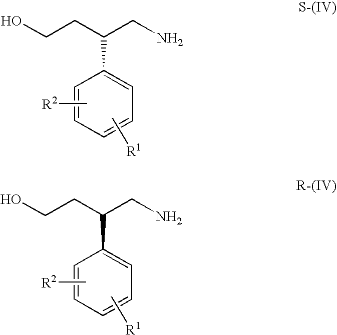

wherein R₁ and R₂ are as defined for the compound of formula (I) above;

by reacting the compounds of formula (IV) with an enantiomerically pure acid in a solvent whereby a mixture of diastereoisomeric salts is obtained; and

(ii) the desired diasteroisomeric salt is separated from the solution.

Step 4

The compound of formula S-(IV) or R-(IV) obtained from Step 3 or the compound of formula (IV) obtained from Step 2 is reacted with a alkyl haloformate of formula R⁹OCOX or an aliphatic carbonate ester of formula R⁹0COOR⁹ in a solvent and in the presence of a base, whereby a compound of formula (V) or S-(V) or R-(V) is obtained:

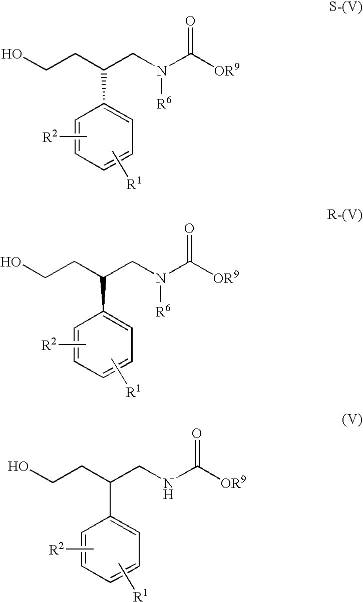

wherein R¹, R², R⁶ and R⁹ are as defined for the compound of formula (I) above and X is chloro or bromo.

Step 5

(i) A compound of formula (V) or S-(V) or R-(V) obtained from Step 4 is reacted with a reducing agent in a solvent whereby a compound of formula (VI) or R-(VI) or S-(IV) is obtained

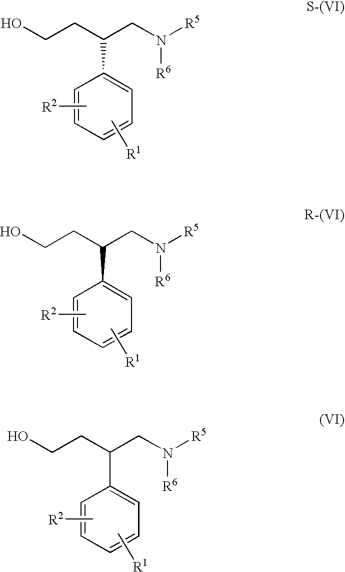

wherein R¹ and R² are as defined for formula (I) above and R¹ and R⁶ are independently selected from hydrogen or methyl, with the proviso that R⁵ and R⁶ are not both methyl; and

(ii) the compound of formula (VI) or S-(VI) or R-(VI) is purified.

Step 6

(i) A compound of formula (VI) or S-(VI) or R-(VI) is reacted with an acid halide of formula R⁸COX in a solvent in the presence of a base; or

a compound of formula (VI) or S-(VI) or R-(VI) is reacted with a compound of formula R⁸COOH in a solvent in the presence of an activating coupling agent, optionally also in the presence of a base;

whereby a compound of formula (VII) or S-(VII) or R-(VII) is obtained

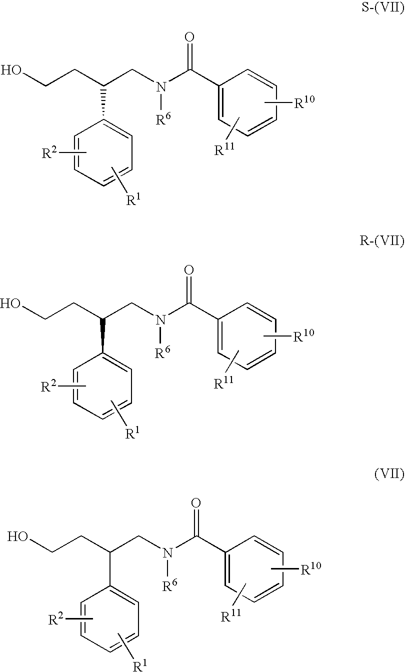

wherein R¹, R², R¹⁰ and R¹¹ are as defined for the compound of formula (I) above, R⁶ is hydrogen or methyl and X is chloro or bromo.

(ii) The compound of formula (VII) or R-(VII) or S-(VII) is purified.

In one embodiment R³ is CH₂NH₂ and R⁴ is hydroxymethyl.

In one embodiment R⁴ is hydroxymethyl and R⁵ or R⁶ is COOR⁹.

In one embodiment R⁴ is hydroxymethyl and R⁵ or R⁶ is COR⁸.

In one embodiment R² is hydrogen and R¹ is bromo, fluoro or iodo.

In one embodiment R⁹ is ethyl.

In one embodiment R⁸ is dibromophenyl or bromo-trifluoromethylphenyl.

In one embodiment the compound of formula (I) is the (S)-enantiomer.

The compounds of formula (I) above as well as intermediates obtained in the process according to the present invention may exist also in the form of technically acceptable salts. Also within the scope of the invention are isomers and stereoisomers of the o compounds of formula (I), as well as of the intermediates thereof, whenever chemically possible.

By “isomers” we mean compounds of formula (I)-(VII), which differ by the position of their functional group (regioisomers) and/or orientation. By “orientation” we mean stereoisomers, diasteroisomers and enantiomers.

The term “amyl” means a linear or branched alkyl group having 5 carbon atoms such as C₅H₁₁.

The term “C₁-C₁₀ alkyl” includes linear or branched alkyl groups having 1 to 10 carbon atoms. Examples of C₁-C₁₀ alkyl include, but are not limited to, methyl, ethyl, propyl, n-propyl, isopropyl, butyl, iso-butyl, sec-butyl, tert-butyl, isoamyl, amyl, hexyl, heptyl, octyl, nonyl and decyl.

The term C₁-C₄ alkyl includes linear or branched alkyl groups having 1 to 4 carbon atoms. Examples of C₁-C₄ alkyl include, but are not limited to, methyl, ethyl, propyl, butyl, isopropyl, tert-butyl and iso-butyl.

The term “cyclic C₃-C₆ alkyl” includes cyclopropyl, cyclobutyl, cyclopentyl, and cyclohexyl.

According to the present invention R¹ may be present either in the 3-, 4- or the 5-position of the aromatic structure of formula (I) and R² may be present at any position in the aromatic structure ortho- or meta- to the substituent R¹ of formula (I). The same relationship is valid for the substituents R¹⁰ and R¹¹ of substituent R⁸ in formula (I).

Examples of suitable haloacetic acid derivatives, which may be used in Step 1 are chloroacetic acid or bromoacetic acid or salts and esters thereof.

The base used in Step 1 may be selected from any one of inorganic carbonates (such as sodium and potassium carbonate); inorganic hydroxides (such as sodium and potassium hydroxide); basic quaternary ammonium salts (such as benzyltrimethylammonium hydroxide); inorganic hydrides (such as lithium, sodium and potassium hydride); alkali metal amides (such as sodium amide); alkali metal diisopropylamides (such as lithium and sodium diisopropylamide); alkali metal hexamethyldisilazides (such as lithium and sodium hexamethyldisilazide); alkali metal alkoxides (such as lithium, sodium and potassium tert-butoxide, lithium, sodium and potassium isopropoxide, lithium, sodium and potassium ethoxide and lithium, sodium and potassium methoxide); amines (such as ammonia, 1,8-diazabicyclo[5.4.0]undec-7-ene, 1,4-diazabicyclo[2.2.2]-octane and 1,8-bis(dimethylamino)-naphthalene); and mixtures thereof.

The solvent used in the reaction of Step 1 may be selected from any one of dimethylsulphoxide; N-methylpyrrolidinone; N,N-dimethylacetamide; N,N-dimethylformamide, sulpholane; tetramethylurea; 1,3-dimethyl-2-imidazolidinone, aromatic hydrocarbons (such as toluene); aliphatic hydrocarbons (such as n-heptane); ethers (such as tetrahydrofuran, 2-methyltetra-hydrofuran, tert-butyl methyl ether and diethyleneglycol dimethyl ether); and chlorinated hydrocarbons (such as dichloromethane t5 and chlorobenzene); amines (such as liquid ammonia, triethylamine, tributylamine, diisopropylamine); and mixtures thereof.

The reaction of Step 1 may also be performed in the presence of a phase-transfer-catalyst selected from any one of a tetraalkylammonium salt; an arylalkylammonium salt; a tetraalkylphosphonium salt; an arylalkylphosphonium salt; a crown ether; an ethylene glycol (e.g. pentaethylene glycol, hexaethylene glycol and polyethylene glycol); and mixtures thereof.

The compound of formula (III) is purified by partition between an organic solvent and an aqueous solution. The organic solvent may be selected from any one of aromatic hydrocarbons (such as toluene, ethylbenzene, cumene and xylene); aliphatic hydrocarbons (such as n-heptane, ligroin, petroleum ether, heptane, hexane, octane, cyclohexane, and cycloheptane); ethers (such as tetrahydrofuran, 2-methyltetrahydrofuran, tert-butyl methyl ether and diethyleneglycol dimethyl ether); chlorinated hydrocarbons (such as dichloromethane and chlorobenzene); lipophilic alcohols (such as n-butanol); and mixtures thereof.

The purification by partition may optionally be followed by further purification using crystallization, i.e. the compound of formula (III) is crystallized from an organic solvent which may be selected from any one of aromatic hydrocarbons (such as toluene, ethylbenzene, cumene and xylene); aliphatic hydrocarbons (such as n-heptane, ligroin, s petroleum ether, heptane, hexane, octane, cyclohexane, and cycloheptane); ethers (such as tetrahydrofuran, 2-methyltetrahydrofuran, tert-butyl methyl ether and diethyleneglycol dimethyl ether); ketones (such as methyl iso-butyl ketone); aliphatic esters (such as ethyl acetate, isopropyl acetate and n-butyl acetate); chlorinated hydrocarbons (such as dichloromethane and chlorobenzene); aliphatic alcohols (such as methanol, ethanol, n-propanol, i-propanol, n-butanol, i-butanol and t-butanol; and mixtures thereof. An antisolvent can optionally be added to obtain a crystalline solid of the compound of formula (III). The antisolvent may be selected from any one of aromatic hydrocarbons (such as toluene, cumene, and xylene); aliphatic hydrocarbons (such as n-heptane, ligroin, petroleum ether, heptane, hexane, octane, cyclohexane, and cycloheptane); and water.

In one embodiment of the present invention Step 1 is carried out at a temperature from −100° C. to +130° C. In one embodiment the reaction is performed at temperatures of from −50° C. to +100° C.

The reaction of Step 2 may be performed according to methods described in March's Advanced Organic Chemistry, Fifth Edition, ISBN 0-471-58589-0, which methods disclose that a compound of formula (III) can be reduced by reacting it, in a solvent, with a reducing agent selected from any one of borane; borane complexes (e.g. borane-tetrahydrofuran-complex, borane-N,N-diethylaniline-complex or borane-dimethyl sulfide-complex); lithium aluminum hydride; sodium aluminum hydride and sodium bis(2-methoxyethoxy)aluminum hydride.

The solvent used for the reaction of Step 2 may be selected from any one of aromatic hydrocarbons (such as benzene, toluene, ethylbenzene, xylene and cumene); aliphatic hydrocarbons (such as cyclohexane, n-heptane, ligroin, petroleum ether, heptane, hexane, octane, cyclohexane, and cycloheptane); ethers (such as tetrahydrofuran, 2-methyltetrahydrofuran, tert-butyl methyl ether and diethyleneglycol dimethyl ether); and mixtures thereof.

The compound of formula (IV) obtained from Step 2 is purified by partition using an organic solvent and an aqueous solution. The organic solution may be selected from any one of aromatic hydrocarbons (such as toluene, cumene and xylene); aliphatic hydrocarbons (such as n-heptane, ligroin, petroleum ether, heptane, hexane, octane, cyclohexane, and cycloheptane); ethers (such as tetrahydrofuran, 2-methyltetrahydrofuran, tert-butyl methyl ether and diethyleneglycol dimethyl ether); aliphatic esters (such as ethyl acetate, isopropyl acetate and n-butyl acetate); chlorinated hydrocarbons (such as dichloromethane and chlorobenzene); lipophilic alcohols (such as n-butanol); and mixtures thereof.

The partition may optionally be followed by crystallization of the product from an organic solvent selected from any one of aromatic hydrocarbons (such as toluene); aliphatic hydrocarbons (such as n-heptane, ligroin, petroleum ether, heptane, hexane, octane, cyclohexane, and cycloheptane); ethers (such as tetrahydrofuran, 2-methyltetrahydrofuran, tert-butyl methyl ether and diethyleneglycol dimethyl ether); aliphatic esters (such as ethyl acetate, isopropyl acetate and n-butyl acetate); chlorinated hydrocarbons (such as dichloromethane and chlorobenzene); aliphatic alcohols (such as methanol, ethanol, n-propanol, i-propanol, n-butanol, i-butanol and t-butanol); and mixtures thereof. An antisolvent can optionally be used to obtain a crystalline solid of the compound of formula (IV). The antisolvent may be selected from any one of aromatic hydrocarbons (such as toluene, cumene and xylene); aliphatic hydrocarbons (such as n-heptane, ligroin, petroleum ether, heptane, hexane, octane, cyclohexane, and cycloheptane); and water.

In one embodiment of the present invention Step 2 is carried out at a temperature of from −70° C. to +130° C. In one embodiment the reaction of Step 2 is performed at a temperature of from 0° C. to +100° C.

The enantiomerically pure acids which are used in the reaction of Step 3 may be selected from any one of mandelic acids (e.g. D- and L-mandelic acid and (R)—O-acetylmandelic acid and (S)-O-acetylmandelic acid, (S)- and (R)-(−)-3-chloro-mandelic acid); (R)- and (S)-methoxy-phenylacetic acid; tartaric acid derivatives (e.g. L- and D-tartaric acid, di-p-toluoyl-L-tartaric acid and di-p-toluoyl-D-tartaric acid, dibenzoyl-L-tartaric acid and dibenzoyl-D-tartaric acid, (−)- and (+)-O,O′-dibenzoyl-1-tartaric acid monodimethylamide, (2R,3R)-tartranilic acid); arylpropionic acids (e.g. (R)- and (S)-naproxen, (R)- and (S)-ibuprofen); phthalic acid derivatives (e.g. (R)- and (S)—N-(1-phenylethyl)-phthalamic acid; other acids such as (S)- and (R)-2-[(phenylamino)-carbonyloxy]propionic acid; (−)-menthoxyacetic acid; L-malic acid; (S)-(+)-citramalic acid; L-pyroglutamic acid; (S)-(−)-2-acetoxy-propionic acid; (S)-(+)-phenylsuccinic acid; phosphoric acid derivatives (e.g. Anicyphos P, Anicyphos N and (S)- and (R)-1,1′-binaphthyl-2,2′-diyl hydrogenphosphate); sulphonic acids (e.g. (+)- and (−)-camphor-10-sulphonic acid); and acids derived from sugars (e.g. (−)-2,3 :4,6-di-o-isopropylidene-2-keto-L-gulonic acid monohydrate).

Solvents useful for Step 3 may be selected from any one of aliphatic alcohols (such as methanol, ethanol, n-propanol, i-propanol, n-butanol, i-butanol and t-butanol); nitriles (such as acetonitrile); ethers such as (tetrahydrofuran, 2-methyltetrahydrofuran, tert-butyl methyl ether and diethyleneglycol dimethyl ether); chlorinated hydrocarbons such as dichloromethane or chlorobenzene; aliphatic esters (such as ethyl acetate, butyl acetate or isopropyl acetate); aromatic hydrocarbons (such as toluene); aliphatic hydrocarbons (such as n-heptane); polar aprotic solvents (such as N-methylpyrrolidinone, N,N-dimethylacetamide or N,N-dimethylformamid); and mixtures thereof. Also, Step 3 may be performed in water or in a solution comprising water and any one of the above-listed organic solvents.

Step 3 is initially performed at temperatures of from 0° C. to the boiling point of the solvent to fully dissolve the components or the formed diastereoisomeric salts. When the components have been dissolved, the temperature of the solution is adjusted to a temperature of from −50° C. to +50° C., to obtain a crystalline salt of the compound of formula R-(IV) or S-(IV). The salt can thereafter be recrystallized from a solvent similar or different to the one used above to improve the optical and chemical purity.

The base useful for the reaction of Step 4 may be selected from any one of aqueous or non-aqueous inorganic bases such as potassium hydroxide; sodium hydroxide; lithium hydroxide; potassium carbonate; sodium carbonate and lithium carbonate.

The organic solvent used in the reaction of Step 4 may be selected from any one of aromatic hydrocarbons (such as toluene, xylene or cumene); aliphatic hydrocarbons (such as n-heptane, ligroin, petroleum ether, heptane, hexane, octane, cyclohexane, cycloheptane and the like); ethers (such as tetrahydrofuran, 2-methyltetrahydrofuran, tert-butyl methyl ether and diethyleneglycol dimethyl ether); aliphatic esters (such as isopropyl acetate); ketones (such as methyl iso-butyl ketone); and chlorinated hydrocarbons (such as dichloromethane and chlorobenzene).

Alternatively, the reaction in Step 4 could also be performed in any of the above-listed organic solvents in the presence of an organic base, which may be selected from any one of pyridine derivatives (lutidines, picolines and pyridine itself); and tertiary amines (such as triethylamine, tributylamine and diisopropylethylamine). Said reaction may be performed in the presence of catalytic 4-(N,N-dimethylamino)-pyridine and/or in the presence of water.

According to one embodiment of the present invention Step 4 is performed at temperatures of from −20° C. to +100° C. In one embodiment of the present invention Step 4 is carried out at temperatures of from 0° C. to +80° C.

The reaction disclosed in Step 5 may be performed according to methods described in March's Advanced Organic Chemistry, Fifth Edition, ISBN 0-471-58589-0, which discloses that a compound of formula (V) or S-(V) or R-(V) can be reduced by reacting said compound with a reducing agent (such as borane; borane complexes (e.g. borane-tetrahydrofuran-complex, borane-N,N-diethylaniline-complex, borane-dimethyl sulfide-complex);lithium aluminum hydride, sodium aluminum hydride and sodium bis(2-methoxyethoxy)aluminum hydride) in the presence of a solvent.

The solvent used for the reaction of Step 5 may be selected from any one of aromatic hydrocarbons (such as benzene, toluene, xylene, ethylbenzene and cumene); aliphatic hydrocarbons (such as cyclohexane, n-heptane, ligroin, petroleum ether, heptane, hexane, octane, cyclohexane and cycloheptane); ethers (such as tetrahydrofuran, 2-methyltetrahydrofuran, tert-butyl methyl ether and diethyleneglycol dimethyl ether ); and mixtures thereof.

The compound of formula (VI) or R-(VI) or S-(VI) obtained from Step 5 may be purified by partition between an organic solvent and an aqueous solution. The organic solvent is selected from any one of aromatic hydrocarbons (such as toluene, cumene, and xylene); aliphatic hydrocarbons (such as n-heptane, ligroin, petroleum ether, heptane, hexane, octane, cyclohexane, and cycloheptane); ethers (such as tetrahydrofuran, 2-methyltetrahydrofuran, tert-butyl methyl ether and diethyleneglycol dimethyl ether); aliphatic esters (such as ethyl acetate, isopropyl acetate and n-butyl acetate); chlorinated hydrocarbons (such as dichloromethane and chlorobenzene); lipophilic alcohols (such as n-butanol); and mixtures thereof.

The partition may optionally be followed by crystallization of the compound of formula (VI) or R-(IV) or S-(IV) from an organic solvent. The organic solvent is selected from any one of aromatic hydrocarbons (such as toluene); aliphatic hydrocarbons (such as n-heptane, ligroin, petroleum ether, heptane, hexane, octane, cyclohexane, and cycloheptane); ethers (such as tetrahydrofuran, 2-methyltetrahydrofuran, tert-butyl methyl ether and diethyleneglycol dimethyl ether); aliphatic esters (such as ethyl acetate, isopropyl acetate and n-butyl acetate); chlorinated hydrocarbons (such as dichloromethane and chlorobenzene); aliphatic alcohols (such as methanol, ethanol, n-propanol, i-propanol, n-butanol, i-butanol and t-butanol); and mixtures thereof. An antisolvent may be used to obtain a purified crystalline solid of the compound of formula (VI) or R-(VI) or S-(VI). The antisolvent is selected from any one of aromatic hydrocarbons (such as toluene, cumene, and xylene); aliphatic hydrocarbons (such as n-heptane, ligroin, petroleum ether, heptane, hexane, octane, cyclohexane, and cycloheptane); and water.

Purification of the compound of formula (VI) or R-(VI) or S-(VI) may alternatively be performed by reacting said compound with a suitable acid to obtain a salt that can be purified by crystallization. Suitable acids for obtaining a salt of said compound may be selected from any one of hydrogen halides (hydrochloric, hydrobromic and hydroiodic acid); sulfuric acid derivatives (such as sulfuric acid, methanesulfonic acid, ethanesulfonic acid, ethanedisulfonic acid, benzenesulfonic acid, and p-toluenesulfonic acid); phosphoric acid; carboxylic acids (such as formic acid, acetic acid, oxalic acid, citric acid, benzoic acid); and any one of the above-listed (see Step 3) chiral acids.

Solvents useful for crystallization of salts obtained from Step 5 may be selected from any one of aliphatic alcohols (such as methanol, ethanol, n-propanol, i-propanol, n-butanol, i-butanol and t-butanol); nitriles (such as acetonitrile); ethers (such as tetrahydrofuran, 2-methyltetrahydrofuran, tert-butyl methyl ether and diethyleneglycol dimethyl ether); chlorinated hydrocarbons (such as dichloromethane or chlorobenzene); aliphatic esters (such as ethyl acetate, butyl acetate or isopropyl acetate); aromatic hydrocarbons (such as toluene); aliphatic hydrocarbons (such as n-heptane); polar aprotic solvents (such as N-methylpyrrolidinone, N,N-dimethylacetamide and N,N-dimethylformamide); and mixtures thereof.

Also, crystallization of salts obtained from Step 5 may also be performed in water or in a solution or suspension between water and any of the above-listed organic solvents. Optionally, an antisolvent selected from an organic solvent or water may be used to obtain a crystalline solid salt of the compound of formula (VI) or R-(VI) or S-(VI). The organic solvent is selected from any one of aromatic hydrocarbons (such as toluene, cumene, and xylene); and aliphatic hydrocarbons (such as n-heptane, ligroin, petroleum ether, heptane, hexane, octane, cyclohexane, and cycloheptane).

In one embodiment Step 5 is carried out at a temperature of from −70° C. to +130° C. In one embodiment, the reaction of Step 5 is performed at a temperature of from 0° C. to +100° C.

The base optionally used in the reaction disclosed in Step 6 may be selected from any one of aqueous or non-aqueous inorganic bases such as potassium hydroxide; sodium hydroxide; lithium hydroxide; potassium carbonate; sodium carbonate; and lithium carbonate.

The organic solvent used in Step 6 may be selected from any one of aromatic hydrocarbons (such as toluene, xylene and cumene); aliphatic hydrocarbons (such as n-heptane, ligroin, petroleum ether, heptane, hexane, octane, cyclohexane, and cycloheptane); ethers (such as tetrahydrofuran, 2-methyltetrahydrofuran, tert-butyl methyl ether and diethyleneglycol dimethyl ether); aliphatic esters (such as isopropyl acetate); and chlorinated hydrocarbons (such as dichloromethane and chlorobenzene).

Alternatively, the reaction in Step 6 may be performed in any of the organic solvents listed above using an organic base. The organic base may be selected from any one of pyridine derivatives (lutidines, picolines and pyridine as such) and tertiary amines (such as triethylamine, tributylamine, diisopropylethylamine). Said reaction may be carried out in the presence of catalytic 4-(N,N-dimethylamino)-pyridine and/or water.

Activating coupling agents for the acid R⁸COOH may be selected from any one of carbodiimides (such as N,N′-dicyclohexylcarbodiimide and 1-(3-dimethylaminopropyl)-3-ethylcarbodiimide hydrochloride); acid chlorides (such as oxalyl chloride and pivaloyl chloride); chloroformates (such as ethyl chloroformate and isobutyl chloroformate); cyanuric chloride; N,N′-carbonyldiimidazole; diethyl chlorophosphite; 2-chloro-1-methyl-pyridinium iodide; and 2,2′-dipyridyl disulphide.

Compounds of formula (VII) or R-(VII) or S-(VII) may be purified by partition between an organic solvent and an aqueous solution. The organic solvent is selected from any one of aromatic hydrocarbons (such as toluene, cumene, and xylene); aliphatic hydrocarbons (such as n-heptane, ligroin, petroleum ether, heptane, hexane, octane, cyclohexane, and cycloheptane); ethers (such as tetrahydrofuran, 2-methyltetrahydrofuran, tert-butyl methyl ether and diethyleneglycol dimethyl ether); ketones (such as methyl iso-butyl ketone); aliphatic esters (such as ethyl acetate, isopropyl acetate and n-butyl acetate); chlorinated hydrocarbons (such as dichloromethane and chlorobenzene); lipophilic alcohols (such as n-butanol) and mixtures thereof. The partition may be followed by crystallization of the compound of formula (VII) or R-(VII) or S-(VII) from an organic solvent selected from any one of aromatic hydrocarbons (such as toluene); aliphatic hydrocarbons (such as n-heptane, ligroin, petroleum ether, heptane, hexane, octane, cyclohexane, and cycloheptane); ethers (such as tetrahydrofuran, 2-methyltetrahydrofiiran, tert-butyl methyl ether and diethyleneglycol dimethyl ether); ketones (such as methyl iso-butyl ketone); aliphatic esters (such as ethyl acetate, isopropyl acetate and n-butyl acetate); chlorinated hydrocarbons (such as dichloromethane and chlorobenzene); aliphatic alcohols (such as methanol, ethanol, n-propanol, i-propanol, n-butanol, i-butanol and t-butanol); and mixtures thereof. An antisolvent may be used to obtain a crystalline solid of the compound of formula (VII) or R-(VII) or S-(VII). The antisolvent is selected from any one of aromatic hydrocarbons (such as toluene, cumene, and xylene); aliphatic hydrocarbons (such as n-heptane, ligroin, petroleum ether, heptane, hexane, octane, cyclohexane, and cycloheptane); and water.

### EXAMPLES

The present invention is described in more detail by the following examples, which do not limit the scope of the present invention.

### Example 1

### Preparation of 3-Cyano-3-(4-fluoro-phenyl)-propionic acid tert-butyl ester (Compound (III-1))

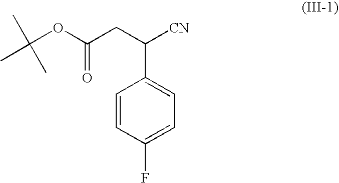

Lithium diisopropylamide (LDA, 52 L, 1.8 M, 93.6 mol) in a solution of Tetrahydrofuran (THF)/Heptane and ethylbenzene was charged to a reactor under a nitrogen atmosphere, and THF (52 L) was then added. The temperature was adjusted to an inner temperature (the temperature of the reaction solution) of −48° C. 4-Fluorophenylacetonitrile (13.0 kg, 96.2 mol) in a THF-solution (25 L) was charged during 1 h and 50 min to the solution comprising LDA, while the temperature of the reaction mixture was kept below −30° C. The temperature was increased to −6° C. over 1 h, during that time the yellow slurry transformed into a dark purple solution. THF (5 L) followed by tert-butylbromoacetate (20.25 kg, 104 mol) and finally THF (25 L) was charged to a second reactor. The temperature was lowered to an inner temperature of −48° C. The dark purple solution above was charged to the tert-butyl-bromoacetate-solution over 7.5 h, while the inner temperature was kept below −34° C. The inner temperature was adjusted to −5° C. and the reaction mixture was quenched by adding a solution of ammonium chloride (12.7 kg) and water (55 L) over 15 min. Methyl tert-butyl ether (MTBE 43 L) was charged and the obtained mixture was stirred for 5 min. After phase separation, the aqueous phase was discarded. Brine (7.6 kg sodium chloride in 25 L of water) was charged to the remaining organic phase and the mixture was stirred for 5 min. The aqueous phase was discarded and the remaining solution was concentrated by distillation at reduced pressure to a volume of 150 L. Isooctane (43 L) was charged and the distillation was continued until the resulting volume was 60 L at which point crystallization started. MTBE (25 L) was charged and the jacket temperature was set to 0° C. After 2 h the batch was filtered (inner temperature 2° C.) and washed with isooctane (2×20 L). After drying 16.8 Kg (72%) of the title compound was obtained.

¹H NMR (DMSO-d₆) δ 7.51 (app d, J=8 Hz, 1 H), 7.50 (app d, J=8 Hz, 1 H), 7.24 (app t, J=8 Hz, 2H), 4.50 (app dd, J₁=6 Hz, J₂=8 Hz, 1 H), 3.02 (app dd, J₁=8 Hz, J₂=16 Hz, 1 H), 2.86 (app dd, J₁=6 Hz, J₂=16 Hz, 1 H), 1.36 (s, 9 H); ¹³C NMR (DMSO-d₆) δ 168.4, 161.7 (d, J_{C,F}=244 Hz), 131.3 (d, J_{C,F}=3 Hz), 129.8 (d, J_{C,F}=9 Hz), 120.6, 115.7 (d, J_{C,F}=22 Hz), 81.0, 39.1, 31.4, 27.6.

### Example 2

### Preparation of 4-Amino-3-(4-fluoro-phenyl)-butan-1-ol (Compound (IV-1))

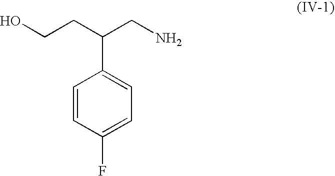

The compound (formula (III-1) obtained from Example 1 (16.7 kg, 67.0 mol) was charged under nitrogen atmosphere to a reactor and THF (50 L) was then added. The temperature was adjusted to an inner temperature of 65° C. Borane-dimethylsulfide complex (16.6 L, 166 mol) in a THF solution (5 L) was charged to the reaction mixture over a period of 43 minutes. The mixture was then refluxed for 2 hours. The reaction mixture was cooled to 10° C. Water (75 L) and hydrochloric acid (25.5 L) was charged to a second vessel and the reaction solution above was charged to this aqueous phase accompanied by gas evolution (H₂ is formed). When the addition was complete (after 1.5 h), the jacket temperature was increased to 105° C. and the solvents were distilled off until the temperature of the reaction mixture reached 85° C. The reaction mixture was refluxed for 12.5 h and then cooled to 24° C. Aqueous sodium hydroxide (50% solution, 32.4 kg) was charged followed by toluene (55 L) and THF (18 L). After phase separation, the aqueous phase was extracted with a mixture of toluene (30 L) and THF (13 L). The organic phases were combined and approximately 65 L of solvent mixture was removed by distillation under reduced pressure. Toluene (40 L) and THF (5 L) was charged to the organic phase and the resulting mixture was clear filtered and returned to the reactor. The solvents were distilled off at reduced pressure until 50 L remained. Toluene (20 L) was charged and the distillation was continued until approximately 35 L remained. The inner temperature was lowered from 59° C. to 12° C. over 1 h and seeding crystals (0.2g) were added, which started the crystallization. Heptane (12 L) was charged and the slurry was cooled down to 6° C. over 2 h. The slurry was filtered and the solid was washed with heptane (2×10 L) and dried. 6.13 kg (50%) of the title compound was obtained.

¹H NMR (DMSO-d₆) δ 7.21 (app d, J=8 Hz, 1 H), 7.19 (app d, J=8 Hz, 1 H), 7.10 (app t, J=8 Hz, 2H), 3.13-3.35 (m, 2 H), 2.59-2.81 (m, 2 H), 1.77-1.94 (m, 2 H), 1.50-1.68 (m, 2 H); ¹³C NMR (CDCl₃) δ 161.7 (d, J_{C,F}=244 Hz), 139.9 (d, J_{C,F}=3 Hz), 129.0 (d, J_{C,F}=8 Hz), 115.6 (d, J_{C,F}=21 Hz), 61.1, 48.2, 46.7, 38.6.

### Example 3

### Preparation of (S)-(−)-4-Amino-3-(4-fluoro-phenyl)-butan-1-ol (R)—O-acetylmandelic acid salt (Compound (S-(IV-1)))

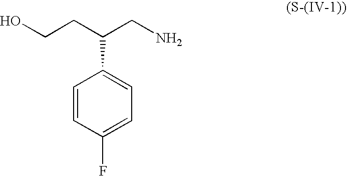

(R)—O-Acetylmandelic acid (18.79 kg, 96.76 mol) was charged to a reactor followed by to water (845 g) and ethyl acetate (EtOAc, 100 L). The solution was stirred at an inner temperature of 17-20° C. for 0.5 h. The clear solution was collected in a drum and the reactor was rinsed with EtOAc (20 L). The rinsing solution was then combined with the above clear (R)—O-acetylmandelic acid solution. 4-Amino-3-(4-fluoro-phenyl)-butan-1-ol (20.64 kg, 112.65 mol) was charged to a reactor followed by absolute ethanol (99.7% w/w, 19 L) and EtOAc (43 L). Stirring was started and the inner temperature was raised to 59° C. The (R)—O-acetylmandelic acid solution was charged to the solution of 4-Amino-3-(4-fluoro-phenyl)-butan-1-ol over 24 min. The dark yellow solution thus obtained started to crystallize at an inner temperature of 53° C. about 5 min after complete addition of (R)—O-acetylmandelic acid. The inner temperature was kept at 52-53° C. for 20 min, and the slurry was then cooled down to 25° C. over 1 h and 20 min. The white slurry was filtered and the solid was washed with EtOAc (2×37.5 L) to give, after drying on the filter, 15.33 kg of needle like white crystals having an optical purity of 83% enantiomeric excess (ee). The ee corrected yield is 66%.

The obtained product (15.33 kg, 40.62 mol) was charged to a reactor followed by absolute 99.5% ethanol (27.5 L) and EtOAc (22.5 L). Stirring was started and the mixture was heated to an inner temperature of 70° C. EtOAc (105 L) was charged to the mixture over 44 min. The inner temperature was kept between 67-70° C. during the addition. The crystallization started 8 min after the last addition of EtOAc (inner temperature 69° C.). The slurry was cooled to an inner temperature of 25° C. over 1 h and 50 min and then filtered. The obtained solid was washed with EtOAc (2×37.5 L) and dried giving 11.65 kg (82% ee corrected yield) of needle-like white crystals having an optical purity of 98% ee according to chiral HPLC.

¹H NMR (DMSO-d₆) δ 7.41 (app dd, J₁=7 Hz, J₂=1 Hz, 2 H), 7.16-7.34 (m, 5 H), 7.12 (app t, J=9 Hz, 2H), 5.53 (app s, 1 H), 3.08-3.33 (m, 2 H), 2.92-3.08 (m, 2 H), 2.78-2.92 (m, 1 H), 2.04 (s, 3 H), 1.77-1.94 (m, 1 H), 1.50-1.69 (m, 1 H); ¹³C NMR (DMSO-d₆) δ 170.6, 169.7, 168.4, 161.1 (d, J_{C,F}=242 Hz), 138.3, 137.7 (d, J_{C,F}=3 Hz), 129.7 (d, J_{C,F}=8 Hz), 127.9, 127.4, 127.3, 115.2 (d, J_{C,F}=21 Hz), 77.2, 58.2, 44.0, 38.7, 36.3, 21.1. [α]_{D} (c 1.0 in methanol, 25° C.) −60.4°.

### Example 4

### (S)-N-[2-(4-Fluorophenyl)-4-hydroxy-butyl]-carbamic acid ethyl ester (Compound (V-1))

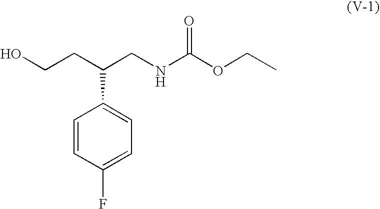

((S)-(−)-4-Amino-3-(4-fluoro-phenyl)-butan-1-ol, (11.61 kg, 30.76 mol) was charged to a stirred solution of aqueous sodium hydroxide (11.30 kg of 50% sodium hydroxide in water, 141.3 mol, diluted to approximately 70 L) at 16° C. inner temperature under nitrogen atmosphere. THF (7.5 L) and toluene (74 L) was charged resulting in a clear two-phase system. The solution was cooled to −1° C. and ethyl chloroformate (3.60 kg, 33.2 mol) in a mixture of THF (1.1 L) and toluene (10 L) was charged to the mixture over 18 min. During the addition the inner temperature rose to 9° C. The reaction mixture was heated to 18° C. over 1 h and 48 min at which point HPLC indicated that the reaction was complete. Toluene (17.5 L) was charged and good mixing was achieved followed by phase separation. The resulting two phases were separated and the aqueous phase was discarded. The organic phase was washed with water (3×8 L) and concentrated to approximately 50 L by distillation at reduced pressure. Toluene (25 L) was charged and the distillation was continued until approximately 30 L of the solvents had been distilled off. Toluene (25 L) was charged and the distillation continued until approximately 40 L remained in. The toluene solution containing the desired product was taken straight into the next Step.

### Example 5

### (S)-(+)-3-(4-Fluorophenyl)4-methylamino-butan-1-ol (Compound (VI-1))

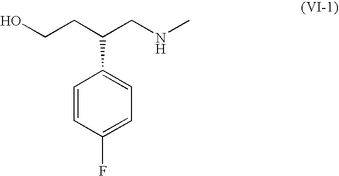

Lithium aluminium hydride (2.11 kg, 55.6 mol) was charged to a reactor containing THF (50 L) at an inner temperature of 20° C. under a nitrogen atmosphere, while stirring. The mixture was heated to an inner temperature of 51° C. and 4 (S)-N-[2-(4-Fluorophenyl)-4-hydroxy-butyl]-carbamic acid ethyl ester in toluene (total volume 43 L) from the previous Step was charged to the lithium aluminium hydride slurry in THF over 2 h. The temperature was kept between 51-68° C. during the addition. The charging vessel was rinsed with toluene (5 L) and the batch was held at 56-58° C. for 2 h after the last addition of 4 (S)-N-[2-(4-Fluorophenyl)-4-hydroxy-butyl]-carbamic acid ethyl ester. The reaction mixture was cooled to an inner temperature of 2 IC and a solution of aqueous sodium bicarbonate (26 L) was charged over 44 min (inner temperature 15° C. and jacket temperature −25° C. at the end of the quench) after which the jacket was adjusted to 20° C. and the batch was left for 15 h. The slurry in the reactor was filtered and the resulting solid was washed with toluene (30 L) in four portions. The filtrate was returned to the reactor (cleaned from aluminium salts) and washed with water (2×10 L) and then clear filtered. The clear filtered solution was returned to the reactor and concentrated to approximately 15 L by distillation under reduced pressure. The distillation was stopped and isooctane (30 L) was charged to the slurry. The slurry was cooled from an inner temperature of 32° C. to 20° C. over 40 min, then filtered and the isolated solid was washed with isooctane (30 L) in four portions. The solid was dried and this resulted in 4.54 kg (75% over two Steps) of the pure title compound.

¹H NMR (DMSO-d₆) δ 7.22 (app d, J=8 Hz, 1 H), 7.20 (app d, J=8 Hz, 1 H), 7.08 (app t, J=8 Hz, 2H), 3.11-3.34 (m, 2 H), 3.72-3.88 (m, 1 H), 3.52-3.66 (m, 2 H), 2.21 (s, 3 H), 1.73-1.91 (m, 1 H), 1.48-1.68 (m, 1 H); ¹³C NMR δ 160.6 (d, J_{C,F}=241 Hz), 140.7 (d, J_{C,F}=3 Hz), 129.3 (d, J_{C,F}=8 Hz), 114.8 (d, J_{C,F}=21 Hz), 58.9, 57.8, 41.3, 37.4, 36.1. [α]_{D} (c 1.0 in methanol, 25° C.) +8.8°.

### Example 6

### (S)-(−)-3,5-Dibromo-N-[2-(4-fluoro-phenyl)-4-hydroxy-butyl]-N-methyl-benzamide (Compound (VII-1))

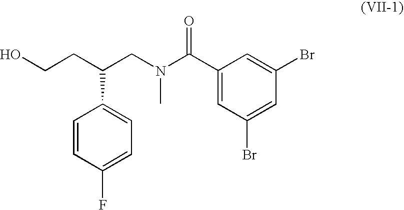

3,5-Dibromobenzoic acid (6.002 kg, 21.44 mol) was mixed with toluene (41.8 kg) and the mixture was stirred under nitrogen. Triethylamine (0.110 kg, 1.09 mol) was added and the temperature was increased to 75° C. jacket temperature. Thionyl chloride (5.172 kg, 43.47 mol) was added continuously over 1 h using a dose pump, which was rinsed with toluene (1.2 kg) after completion of the addition. The reaction mixture was stirred at 73° C. jacket temperature for 12 h and then the temperature was decreased to an inner temperature of 30° C. before sampling. HPLC analysis indicated complete conversion to the acid chloride and the mixture was then evaporated to dryness. The isolated 3,5-dibromobenzoyl chloride was dissolved in toluene (11.44 kg) and the formed solution was evaporated to dryness again at a jacket temperature of 40° C.

Sodium hydroxide (3.49 kg of a 49% aqueous solution, 42.8 mol) was mixed with water (24.0 kg) in a reactor. A nitrogen atmosphere was established and (S)-(+)-3-(4-Fluorophenyl)-4-methylamino-butan-1-ol (4.01 kg, 20.3 mol) was added. The resulting mixture was agitated at a jacket temperature of 18° C. The isolated 3,5-dibromobenzoyl chloride from above was dissolved in toluene (30.7 kg) using a jacket temperature of 29° C. and the resulting solution was added to the slurry of (S)-(+)-3-(4-Fluorophenyl)-4-methylamino-butan-1-ol in aqueous sodium hydroxide over 2 h at an inner temperature of between 22° C. and 27° C., to give a yellowish emulsion. After complete addition the addition vessel was rinsed with toluene (8.00 kg) and this solvent was added to the reaction mixture, which was then agitated at a jacket temperature of 20° C. for another 30 min. HPLC analysis of the organic layer indicated complete conversion (>99%) and the aqueous phase was separated off. Water (11.2 kg) was added and the resulting two-phase system was agitated for about 10 min. The aqueous phase was separated off and the organic layer was washed in the same way as described above using a second portion of water (12.1 kg). The organic layer was evaporated to dryness using a jacket temperature of 40° C. giving 10.9 kg of title compound having a chromatographic purity of 98.9% and an assay of 90.8% according to proton-NMR (assay-corrected yield 106%). The main volatile impurities were toluene 5.9% w/w and water 0.2% w/w. The optical purity according to HPLC was 99% ee.

¹H NMR (spectrum complicated by restricted rotation around the amide bond, DMSO-d₆) δ 7.89 and 7.84 (two singlets appearing as a doublet with J=22 Hz, 1 H), 7.29-7.40 (m, 1 H), 7.20 (app s, 1 H), 6.98-7.24 (m, 3 H), 6.94 (app s, 1 H), 4.37-4.47 (m, 1 H), 3.53-3.77 (m, 1 H), 3.00-3.46 (m, 4 H), 2.65 and 2.96 (methyl group on nitrogen appears as two separate singlets, totally 3 H), 1.54-1.89 (m, 1 H), ¹³C NMR δ (spectrum complicated by restricted rotation around at least the amide bond, DMSO-d₆) 167.2 and 167.0 (carbonyl carbon), 161.0 (d, J_{C,F}=243 Hz), 140.5, 140.0, 138.4,137.8, 134.0, 133.8, 129.85, 129.79, 129.73, 129.67, 128.1 (d, J_{C,F}=8 Hz), 122.5, 122.2, 115.1, 115.0, 114.8, 58.42, 58.35, 51.9, 39.8, 37.4, 36.2, 35.2, 33.0; [α]_{D} (c 1.0 in methanol, 25° C.) −11.4°.

### Example 7

### 3-bromo-N-[(2S)-2-(4-fluorophenyl)-4-hydroxybutyl]-N-methyl-5-(trifluoromethyl)benzamide (Compound (VII-2))

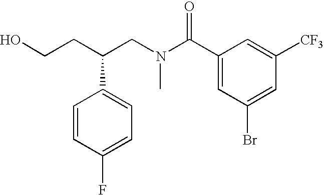

Under a nitrogen atmosphere 39.9 g (148 mmol) of 3-bromo-5-(trifluoromethyl)benzoic acid, 400 mL of toluene, and 1.1 mL of triethylamine (7.9 mmol) were charged to a 1L flask. The mixture was stirred to obtain a solution and warmed to 75° C. 27.2 g (229 mmol) of thionyl chloride was added over 20 minutes resulting in a pale yellow solution. After complete addition the temperature was increased to 100° C. After 1 hour the mixture was sampled and conversion found to be complete. Excess thionyl chloride and some solvent (total 145 mL) were distilled off at 30° C. using a water aspirator. Fresh solvent (160 mL) was added, and again partially removed by distillation. The resulting acid chloride solution (3-bromo-5-(trifluoromethyl)benzoyl chloride, 320 mL) was transferred to a dropping funnel. The reaction flask was filled with 28.3 g of (3S)-3-(4-fluorophenyl)-4-(methylamino)butan-1-ol (143.5 mmol), 145 mL of water and 12.5 g of sodium hydroxide (313 mmol) with some cooling. Starting at 20° C. the solution of acid chloride, 3-bromo-5-(trifluoromethyl)benzoyl chloride, was added at such a rate (˜20 mins) that the temperature was kept below 30° C. The reaction was continued for 30 minutes, then checked for conversion and worked up. The phases were separated. The toluene solution was washed once at 55° C. with 100 mL of water. The turbid organic phase was dried by azeotropic distillation under reduced pressure. In addition some solvent was removed. The final obtained amount of solution was 125.5 g. This was split in two equal parts. One part was used directly for the next reaction. The second part was used to isolate the pure product. 62.8 g of solution was concentrated to 32.5 g of a brown liquid. This was then chromatographed over 450 g of silica, and eluted with dichloromethane with some methanol as modifier (gradually increase from 1% to 3%). Several fractions with in total 25.9 g (57.8 mmol) of product, 3-bromo-N-[(2S)-2-(4-fluorophenyl)-4-hydroxybutyl]-N-methyl-5-(trifluoromethyl)benzamide, as a yellowish viscous oil with a purity of 96.5% or better (by GC-MS) were collected. This corresponds to a yield of 80.5%.

¹H NMR (spectrum complicated by restricted rotation around the amide bond, chloroform-d) δ 7.75 (apparent singlet, 1 H), 6.73-7.51 (m, 6 H), 2.83-3.92 (m, 5 H), 3.08 and 2.67 (methyl group on nitrogen appears as two separate singlets, totally 3 H), 1.42-2.06 (m, 3 H), ¹³C NMR δ (spectrum complicated by restricted rotation around at least the amide bond, chloroform-d) 168.8 and 168.5 (carbonyl carbon), 161.9 (d, J_{C,F}=246 Hz), 139.0, 138.6, 137.4, 136.2, 133.2, 133.1, 132.6 (apparent quartet, J_{C,F}=34 Hz), 129.5, 129.4, 129.3, 126.8, 124.1, 122.9, 122.7, 122.3, 121.3, 118.6, 116.0, 115.7, 115.5, 59.9, 59.6, 57.8, 53.5, 53.1, 39.8, 39.6, 38.3, 36.5, 35.5, 33.5; MS 450 and 448 (M+1, Br isotope pattern); [α]_{D} (c 1.0 in methanol, 20° C.) −5.6°.

1. A process for the preparation of a compound of formula (I): 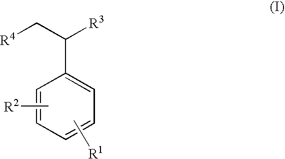
wherein
R¹ is selected from fluoro, bromo, iodo, C₁-C₁₀ alkyl, phenyl, C₃-C₆ cycloalkyl, trifluoromethyl, difluoromethyl and fluoromethyl;
R² is selected from hydrogen, fluoro, bromo, iodo C₁-C₁₀ alkyl, trifluoromethyl, difluoromethyl and fluoromethyl;
R³ is CH₂NR⁵R⁶;
R⁴ is hydroxymethyl;
R⁵ and R⁶ are independently selected from hydrogen, methyl, COR⁸ and COOR⁹;
R⁸ is 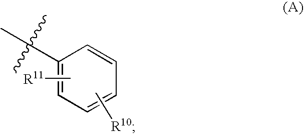
R⁹ is selected from C₁-C₄ alkyl;
R¹⁰ is selected from fluoro, chloro, bromo, iodo, C₁-C₁₀ alkyl, phenyl,
C₃-C₆ cycloalkyl, trifluoromethyl, difluoromethyl and fluoromethyl;
R¹¹ is selected from fluoro, chloro, bromo, iodo, C₁-C₁₀ alkyl, phenyl and C₃-C₆ cycloalkyl;
with the proviso that R⁵ and R⁶ are not the same unless both R⁵ and R⁶ are hydrogen; and
with the further proviso that if one of R⁵ or R⁶ is COR⁸ then the other is methyl; and
with the further proviso that if one of R⁵ or R⁶ is COOR⁹, then the other is hydrogen;
comprising the following steps:
(i) reacting a compound of formula (III) 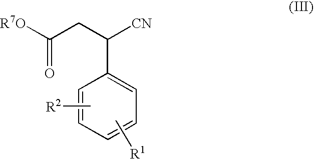
wherein
R⁷ is selected from tert-butyl, iso-butyl, iso-propyl and iso-amyl with a reducing agent in a solvent whereby a compound of formula (I) is provided; 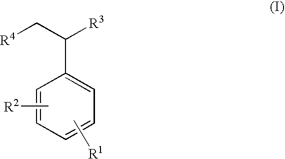
wherein R¹, R², R³ and R⁴ of formula (III) and (I) respectively are as defined above;
(ii) optionally purifying the compound of formula (I).

2. The process according to claim 1, further comprising a step of resolution of the compound of formula (I) into its (R)- and (S)-enantiomers, wherein the compound of formula (I) is reacted with an enantiomerically pure acid in the presence of a solvent, whereby a mixture of diastereoisomeric salts is obtained; and the desired diasteroisomeric salt is separated from the mixture.

3. The process according to claim 1, wherein Steps (i) and (ii) are carried out at a temperature of from −70° C. to +130° C.

4. The process according to claim 3, wherein Steps (i) and (ii) are carried out at a temperature of from 0° C. to 100° C.

5. The process according to claim 2, wherein the step of resolution is carried out at a temperature of from −50° C. to the boiling point of the solvent.

6. The process according to any one of claims 1-5, wherein the solvent used for Step (i) is selected from aliphatic alcohols, nitriles, ethers, chlorinated hydrocarbons, aliphatic esters, aromatic hydrocarbons and water; and mixtures thereof.

7. The process according to claim 6, wherein the solvent is selected from tetrahydrofuran; 2-methyltetrahydrofuran; tert-butyl methyl ether; and diethyleneglycol dimethyl ether.

8. The process according to claim 6, wherein the solvent is ethylacetate and ethanol.

9. The process according to one of claim 2 or 5, wherein the enantiomerically pure acid used is selected from mandelic acids; (R)- and (S)-methoxy-phenylacetic acid; tartaric acid derivatives; arylpropionic acids; phthalic acid derivatives; (S)- and (R)-2-[(phenylamino)-carbonyloxy]propionic acid; (−)-menthoxyacetic acid; L-malic acid; (S)-(+)-citramalic acid; L-pyroglutamic acid;(S)-(−)-2-acetoxy-propionic acid; (s)-(+)-phenylsuccinic acid; phosphoric acid derivatives; sulphonic acids; and acids derived from sugars.

10. The process according to claim 9, wherein the enantiomerically pure acid is selected from D-mandelic acid; L-mandelic acid; (R)—O-acetylmandelic acid; (S)—O-acetylmandelic acid; (S)-(−)-3-chloro-mandelic acid; and (R)-(−)-3-chloro-mandelic acid; and (R)-Naproxen (S)-Naproxen; Anicyphos P and Anicyphos N.

11. A compound of formula (I): 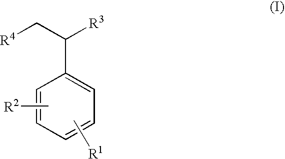
or a technically acceptable salt, solvate or steroisomer thereof, wherein
R¹ is selected from fluoro, bromo, iodo, C₁-C₁₀ alkyl, phenyl, C₃-C₆ cycloalkyl, trifluoromethyl, difluoromethyl and fluoromethyl;
R² is selected from hydrogen, fluoro, bromo, iodo, C₁-C₁₀ alkyl, trifluoromethyl, difluoromethyl and fluoromethyl;;
R³ is CH₂NR⁵R⁶;
R⁴ is hydroxymethyl;
R⁵ and R⁶ are independently selected from hydrogen, methyl, COR⁸ and COOR⁹;
R⁸ is 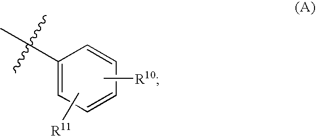
R⁹ is selected from C₁-C₄ alkyl;
R¹⁰ is selected from fluoro, chloro, bromo, iodo, C₁-C₁₀ alkyl, phenyl, C₃-C₆ cycloalkyl, trifluoromethyl, difluoromethyl and fluoromethyl;
R¹¹ is selected from fluoro, chloro, bromo, iodo, C₁-C₁₀ alkyl, phenyl and C₃-C₆ cycloalkyl;
with the proviso that R⁵ and R⁶ are not the same unless both R⁵ and R⁶ are hydrogen; and
with the further proviso that if one of R⁵ or R⁶ is COR⁸ then the other is methyl; and
with the further proviso that if one of R⁵ or R⁶ is COOR⁹, then the other is hydrogen;
with the exception of:
4-amino-3-(3,4-difluorophenyl)-1-butanol;
3-(3,4-difluorophenyl)-4-(ethoxycarbonylamino)-1-butanol;
3-(3,4-difluorophenyl)-N-methyl-4-amino-1-butanol;
3-(4-fluorophenyl)-4-(methylamino)butan-1-ol;
3-(4-bromophenyl)-4-(methylamino)butan-1-ol and
4-(methylamino)-3-(4-methylphenyl)butan-1-ol.

12. The compound according to claim 11, wherein R³ is CH₂NH₂ .

13. The compound according to claim 11, wherein R⁵ or R⁶ is COOR⁹.

14. The compound according to claim 13, wherein R⁹ is C₁-C₄ alkyl.

15. The compound according to claim 14, wherein R⁹ is ethyl.

16. The compound according to any one of claims 11-15, wherein R⁸ is dibromophenyl or bromo-trifluoromethylphenyl.

17. The compound according to claim 11, wherein R⁵ or R⁶ is COR⁸.

18. The compound according to any one of claims 11-15 and 17, wherein R² is hydrogen and R¹ is selected from fluoro, bromo or iodo.

19. The compound according to claim 18, wherein R¹ is fluoro.

20. The compound according to any one of claims 11-15 and 17, wherein the compound of formula (I) is the (S)-enantiomer.

21. The compound according to claim 11, having the formula [chemical structure]
